(19) United States
(12) Patent Application Publication
(10) Pub. No.: US 2020/0015750 A1
(43) Pub. Date: Jan. 16, 2020

(54) NEEDLE ASSEMBLY WITH TEMPERATURE MEASUREMENT DEVICE

(71) Applicant: PATRIOT WORLDWIDE, INC., Holliston, MA (US)

(72) Inventors: Michael Pike, Acushnet, MA (US); Herb Dwyer, Alburuerque, NM (US)

(21) Appl. No.: 16/508,432

(22) Filed: Jul. 11, 2019

Related U.S. Application Data
(60) Provisional application No. 62/696,521, filed on Jul. 11, 2018.

(51) Int. Cl. A61B 5/00; A61B 5/01

(52) U.S. Cl. CPC A61B 5/6848; A61B 5/01; A61B 2562/227; A61B 2562/043; A61B 2562/222; A61B 2562/0271

(57) ABSTRACT

A needle assembly includes an elongated, hollow, hypodermic needle and a temperature measurement device at least partially disposed within the needle. The temperature measurement device comprises a plurality of parallel thermal sensors collectively retained in a fixed relationship within a common dielectric sheath. Each thermal sensor includes a temperature-sensing region which is capable of generating a voltage in proportion to the sensed temperature. The temperature-sensing regions for the plurality of thermal sensors are arranged in an offset relationship within the hypodermic needle, with each temperature-sensing region located at a different position relative to the sharpened tip of the hypodermic needle. Accordingly, in use, needle assembly is designed to compile temperature measurements at multiple locations along the length of the needle, thereby yielding a comprehensive and accurate temperature profile of the body into which the hypodermic needle is inserted.

### CROSS-REFERENCE TO RELATED APPLICATIONS

The present application claims the benefit under 35 U.S.C. 119(e) of U.S. Provisional Patent Application No. 62/696,521, which was filed on Jul. 11, 2018 in the names of Michael Pike et al., the disclosure of which is incorporated herein by reference.

### FIELD OF THE INVENTION

The present invention relates generally to hypodermic needles and, more particularly, to hypodermic needles with temperature measurement capabilities.

### BACKGROUND OF THE INVENTION

Hypodermic needles are well known in the art and are used in a wide variety of applications including, but not limited to, medical research, surgery and drug delivery. A hypodermic needle, or probe, is typically constructed a thin, hollow tube with a sharpened tip at one end to facilitate insertion into the body.

In certain medical applications, it has been found that the monitoring of temperature within the surrounding insertion area is essential to the effectiveness of a particular procedure. For this reason, it is known in the art for hypodermic needles to be incorporated with a single temperature measurement device, such as a thermocouple, to monitor the temperature of the immediate needle-insertion region.

Although well known in the art, hypodermic needles with temperature measurement capabilities have been found to suffer from a notable drawback. Specifically, the temperature data accumulated by conventional hypodermic needles is limited to the insertion area surrounding the temperature sensor and is therefore largely dependent upon on the specific depth of insertion of the needle into the body. Because certain medical procedures are reliant on a high degree of temperature measurement accuracy, the imprecise collection of temperature data can have adverse consequences.

To remedy this shortcoming, multiple hypodermic needles, each with temperature measurement capabilities, are often concurrently disposed at various depths within a body to compile an array of temperature data that, in turn, is used to provide a more comprehensive temperature profile of the body into which the needles are inserted. This expansion of data is therefore utilized to provide greater temperature measurement accuracy, which often results in the greater overall success of the underlying procedure.

Although desirable for the reasons set forth above, it is to be understood that the use of multiple temperature-measuring needles introduces a number of notable drawbacks.

As a first drawback, the use of multiple temperature-measuring needles renders certain medical procedures more complex, time consuming and inefficient to perform.

As a second drawback, the use of multiple, separate, temperature-measuring needles requires acute, manual precision in the relative depth that each needle is inserted. Due to the unknown distance between adjacent temperature sensors, any imprecision in needle insertion depth introduces potential errors in the resultant temperature profile.

As a third drawback, the use of multiple, separate, temperature-measuring needles often creates considerable variance in the response time between the various temperature sensors (e.g. due to sizable discrepancies in signal travel length). Because certain medical procedures are reliant upon immediate temperature measurements, any delay in response time can potentially compromise procedure effectiveness.

As a fourth drawback, the use of multiple temperature-measuring needles directly increases the amount of disruption, and any resultant pain, to the region of the body into which they are insertion. For this reason, the amount of probing into a body is typically minimized to the greatest extent possible.

### SUMMARY OF THE INVENTION

It is an object of the present invention to provide a new and improved needle assembly.

It is another object of the present invention to provide a new and improved needle assembly with temperature measurement capabilities.

It is yet another object of the present invention to provide a needle assembly of the type as described above which provides a comprehensive and highly accurate temperature profile of the body into which it is inserted.

It is still another object of the present invention to provide a needle assembly of the type as described above which has a limited number of parts, is inexpensive to manufacture and is easy to use.

Accordingly, as one feature of the present invention, there is provided a needle assembly comprising (a) an elongated, hollow needle having a first end and a second end, and (b) a temperature measurement device at least partially disposed within the needle, the temperature measurement device comprising a plurality of thermal sensors, each thermal sensor comprising a temperature-sensing region, (c) wherein the temperature-sensing regions for the plurality of thermal sensors are located within the hollow needle at different positions relative to the first end.

Various other features and advantages will appear from the description to follow. In the description, reference is made to the accompanying drawings which form a part thereof, and in which is shown by way of illustration, an embodiment for practicing the invention. The embodiment will be described in sufficient detail to enable those skilled in the art to practice the invention, and it is to be understood that other embodiments may be utilized and that structural changes may be made without departing from the scope of the invention. The following detailed description is therefore, not to be taken in a limiting sense, and the scope of the present invention is best defined by the appended claims.

### BRIEF DESCRIPTION OF THE DRAWINGS

In the drawings, wherein like reference numerals represent like parts:

FIGS. 1(a) and 1(b) are top perspective and top plan views, respectively, of a needle assembly constructed according to the teachings of the present invention;

FIG. 2 is an exploded perspective view of selected components of the needle assembly shown in FIG. 1(a);

FIG. 3 is an enlarged, fragmentary, top view of the needle assembly shown in FIG. 1(b), with selected internal components of the needle assembly being shown in dashed form to illustrate the configuration upon assembly; and

FIGS. 4(a)-(c) are fragmentary, front perspective, front plan and top plan views, respectively, of one of the temperature sensors shown in FIG. 2.

### DETAILED DESCRIPTION OF THE INVENTION

### Needle Assembly 11

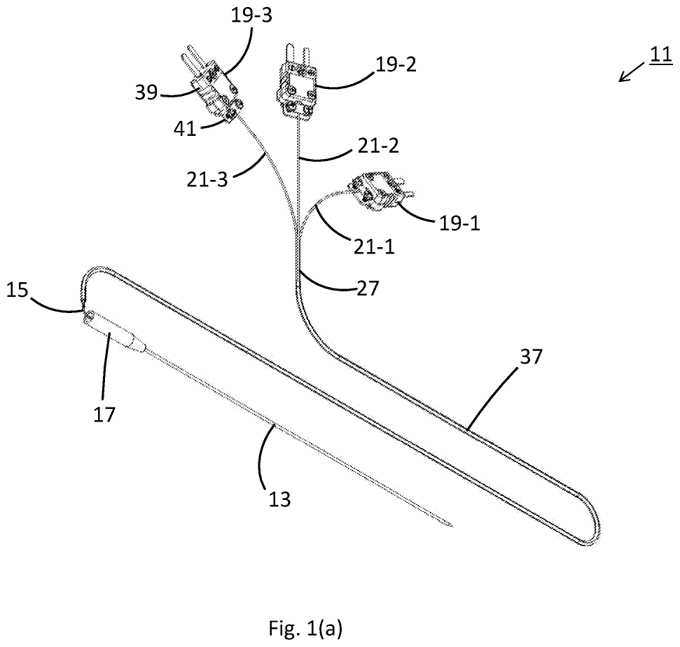

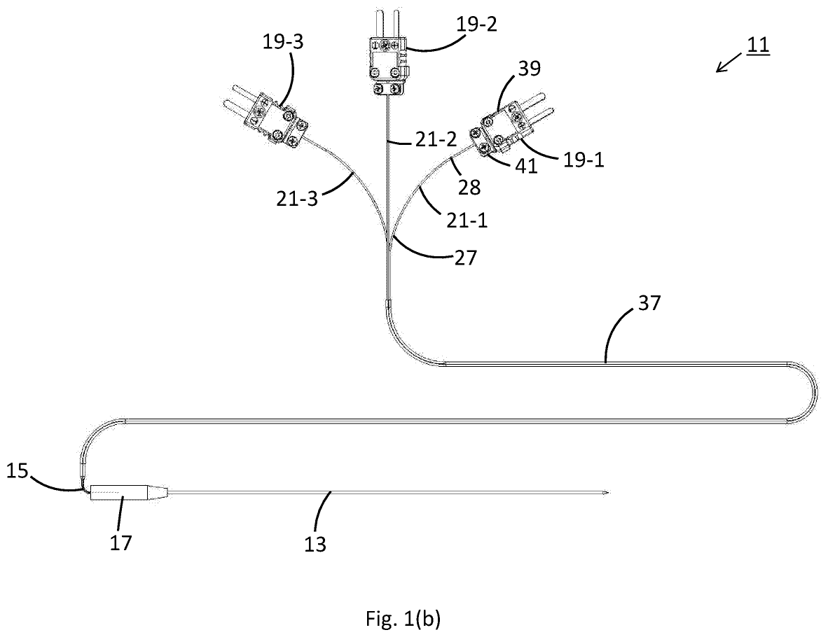

Referring now to FIGS. 1(a) and 1(b), there is shown a needle assembly constructed according to the teachings of the present invention, the needle assembly being identified generally by reference numeral 11. As will be explained in detail below, needle assembly 11 is designed to measure temperatures at multiple locations along its length, thereby rendering needle assembly 11 particularly useful in connection with certain medical applications.

Although needle assembly 11 is described herein principally for use in medical applications, such as in surgical procedures, it is to be understood that needle assembly 11 need not be limited to any particular use. Rather, it is to be understood that the multi-sensor construction of needle assembly 11 could be modified, as needed, for implementation in a wide variety of different applications without departing from the spirit of the present invention.

As can be seen, needle assembly 11 comprises (i) a hypodermic needle 13, (ii) a multi-sensor, temperature measurement device 15 incorporated into hypodermic needle 13, (iii) a needle hub 17 mounted onto hypodermic needle 13 through which electrical leads for device 15 externally extend, and (iv) a plurality of connectors 19-1 thru 19-3 mounted on the electrical leads for device 15 in electrical communication therewith.

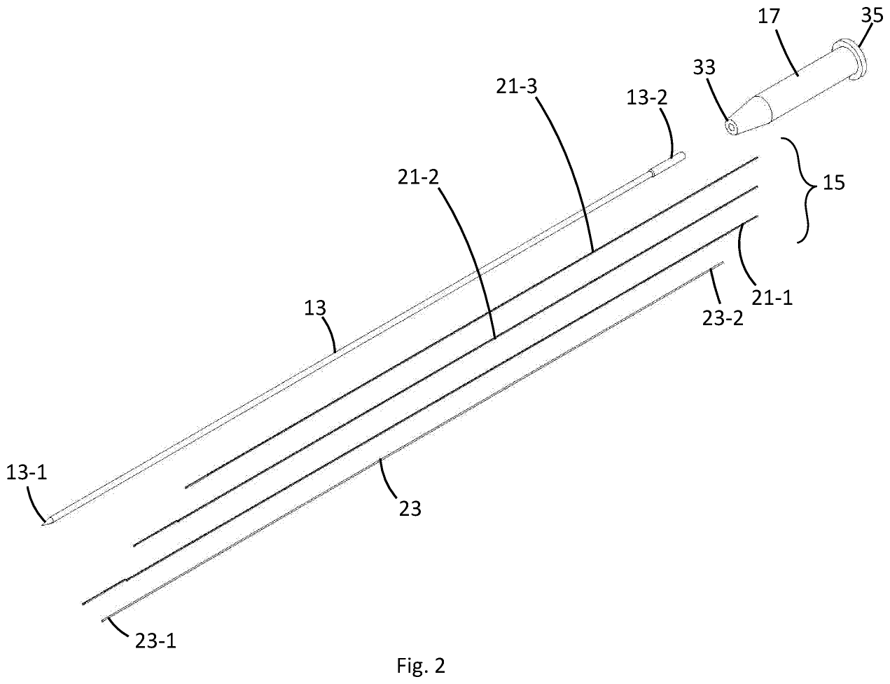

As seen most clearly in FIG. 2, hypodermic needle 13 is constructed as an elongated, thin, hollow tube formed of a suitable material, such as stainless steel. Needle 13 is preferably provided with a sharpened tip 13-1 at one end to facilitate insertion into a body and an opened port 13-2 at its opposite end to provide access into its longitudinal, interior channel.

As defined herein, needle 13 represents any conventional hollowed needle, or probe, which is suitably dimensioned to axially receive temperature measurement device 15 therewithin. For example, needle 13 may be of the type commonly used in connection with various medical procedures and related applications.

A multi-sensor, temperature measurement device 15 is incorporated into hollow needle 13, as will be explained further in detail below. The multi-sensor construction of device 15, as well as its integration into hypodermic needle 13, affords needle assembly 11 with the ability to compile multiple, concurrent temperature readings at various locations along its length, which serves as a principal novel feature of the present invention. As a result, needle assembly 11 is uniquely capable of providing a comprehensive and accurate temperature profile of the body into which it is inserted.

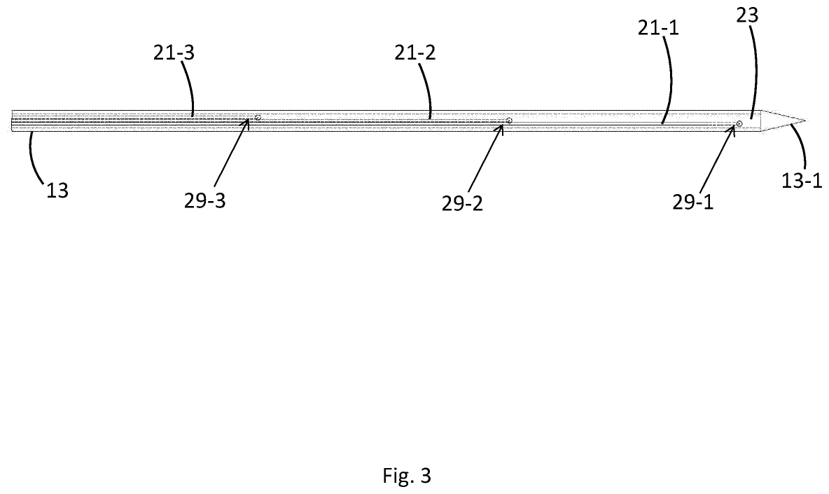

Temperature measurement device 15 comprises (i) a plurality of independent temperature sensors 21-1, 21-2 and 21-3, and (ii) a tubular plastic sheath 23 into which sensors 21 are fittingly, coaxially disposed. As part of the assembly process, which will be described further below, sheath 23, with sensors 21 disposed therein, is inserted into hollow needle 13 to render needle assembly 11 a unitary device, as seen most clearly in FIG. 3.

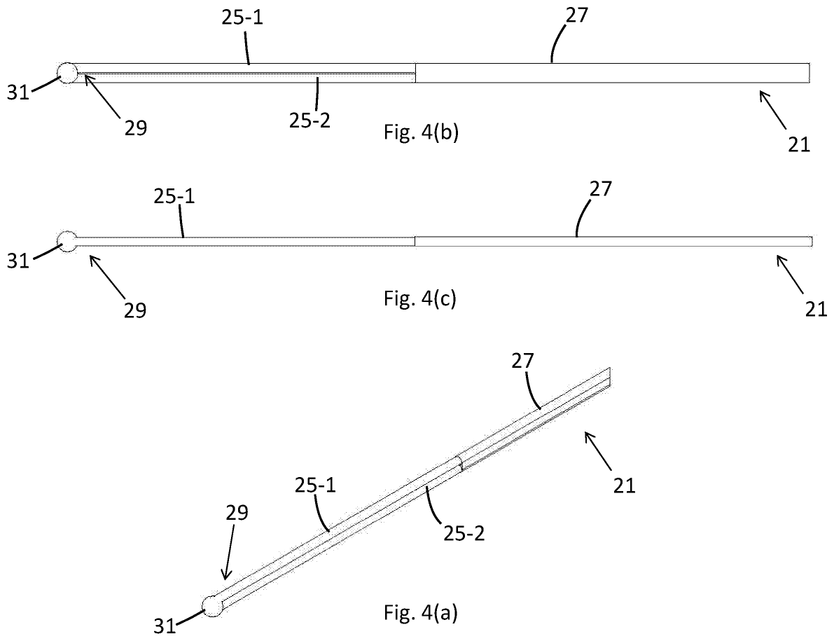

Referring now to FIGS. 4(a)-(c), each temperature, or thermal, sensor 21 is represented herein as comprising a first and second sensor wires 25-1 and 25-2 of dissimilar metals which are dielectrically isolated, or separated, through the application of a layer of insulation 27 over the majority of their lengths.

In order to minimize space requirements within needle 13, each sensor wire 25 is applied with insulation 27 through a multi-pass bath dipping process to achieve insulation thickness in the order of tens of thousandths of an inch. The provision of insulation 27 is critical to prevent the formation of electrical junctions along the intermediary region of sensor 21, thereby improving its overall measurement robustness.

At the external, or distal, end 28 of insulation 27, sensor wires 25 are exposed and, in turn, electrically coupled together with a corresponding connector 19, as shown in FIG. 1(b). Furthermore, as shown in FIGS. 4(a)-(c), the exposed (i.e. non-insulated) section of sensor wires 25 that is located within needle 13 is, in turn, joined together at its free ends to form a primary temperature-sensing junction, or region, 29.

In the present embodiment, a weld bead 31 is shown connecting sensor wires 25 together to form temperature-sensing junction 29. However, alternative means for coupling sensor wires 25 to form junction 29 could be achieved (e.g. by simply twisting wire leads together) without departing from the spirit of the present invention. Preferably, once a temperature-sensing junction 29 is formed, a thin layer of UV cured adhesive (not shown) is applied to and coats junction 29 to ensure adequate structural integrity.

As can be appreciated, junction 29 serves as the primary temperature-sensing region for each sensor 21. More specifically, in accordance with the well-known thermoelectric effect, also commonly referred to as the “Seebeck Effect,” the dissimilar alloys for wires 25 form an electrical voltage in proportion to the temperature differential on each side of temperature-sensing junction 29 (i.e. the temperature within the immediate region of the needle insertion body as compared to the temperature within the thermowell defined within the interior of sheath 23).

It should be noted that each temperature sensor 21 is not limited to the specific, above-described, thermocouple-type construction. Rather, it is to be understood that temperature sensor 21 represents any thermoelectric device that is commonly utilized to measure temperature, including thermoresistors, resistance temperature detectors (RTD) and the like, without departing from the spirit of the present invention.

Referring back to FIG. 3, the three temperature sensors 21-1, 21-2 and 21-3 are arranged in parallel, with temperature-sensing junctions 29-1, 29-2 and 29-3, respectively, offset or staggered at equidistantly spaced locations within the length of needle 13. In this manner, temperature data can be collected at various points along the length of needle 13, which is highly desirable.

To fix the relative position of temperature-sensing junctions 29, an ultraviolet (UV) cured adhesive is preferably applied between insulation 27 of adjacent (i.e. successive) temperature sensors 21. In this manner, the adhesive permanently secures together the plurality of individual sensors 21, thereby enabling each successive temperature-sensing junction 29 to be retained within needle 13 at a fixed, specified distance from needle tip 13-1, thereby providing greater temperature measurement accuracy.

It should be noted that needle assembly 11 is not limited to the particular number and arrangement of temperature sensors 21 shown herein. Rather, it is to be understood that the number and relative position of sensors 21 within needle 13 could be modified, as needed, without departing from the spirit of the present invention.

As seen in FIG. 2, sheath 23 is constructed as an elongated, thin, hollow tube formed of a suitable dielectric material, such as a polymer material. Sheath 23 is preferably provided with an enclosed first end 23-1, which is dimensioned to align fittingly within tip 13-1 of hypodermic needle, and an open second end 23-2. Accordingly, once temperature sensors 21 are secured together with adhesive, thereby fixing the relative locations of temperature-sensing junctions 29, the resultant assembly is axially inserted into the interior channel of sheath 23. Thereafter, additional UV-cured adhesive is applied to open end 23-2 of sheath 23 to ensure that sensors 21 and, in particular, temperature-sensing junctions 29 do not shift within sheath 23 (as well as needle 13) during subsequent use and handling of needle assembly 11. In this manner, dielectric sheath 23 not only facilitates construction of needle assembly 11 but also serves as a protective layer that prevents accidental grounding of the individual thermal sensors 21 via needle 13.

Several dimensional factors, including the total number of temperature sensors 21, the gage of each sensor wire 25, the thickness of insulation 27 as well as the inner and outer diameters of sheath 23 and needle 13, are preferably calculated in order to minimize the gap, or spacing, between temperature sensors 21 and sheath 23, as well as between sheath 23 and needle 13. Minimizing gaps not only reduces the overall cross-section of needle 13 but also improves the response time and overall measurement accuracy of thermal sensors 21.

As seen in FIG. 2, a needle hub 17 is preferably constructed as an enlarged, unitary, hollow tubular component formed of a rigid and durable material, such as plastic. Hub 17 comprises a reduced-diameter proximal section 33, which is dimensioned to fittingly receive port 13-2 of hypodermic needle 13, and an enlarged distal section 35 through which the electrical leads for thermal sensors 21 exit. Among other functions, needle hub 17 can be used to facilitate actuation of needle 13.

### Assembly of Needle Assembly 11

The construction of needle assembly 11 as set forth above not only provides certain functional advantages (e.g. enhanced temperature profile capabilities) but also facilitates its assembly. Most notably, for ease of assembly and optimized performance, temperature-measurement device 15 is preferably assembled in its entirety prior to insertion into hypodermic needle 13, thereby ensuring that construction specifications are accurately met.

In other words, as referenced above, temperature sensors 21 are arranged in parallel such that the plurality of temperature-sensing junctions 29 are offset, or staggered, at predefined locations. Then, to fix the relative position of temperature-sensing junctions 29, a UV-cured adhesive is applied between insulation 27 of adjacent temperature sensors 21. In this manner, the adhesive permanently secures together the plurality of individual sensors 21.

In turn, the plurality of adhesively-bonded, thermal sensors 21 is inserted into the interior channel of sheath 23. Thereafter, additional UV-cured adhesive is applied to open end 23-2 of sheath 23. The entire pre-assembled sheath 23 (i.e. with sensors 21 mounted therein) is inserted through hub 17 and into needle 13, which is initially separate from hub 17.

The pre-assembled sheath 23 is advanced into needle 13 until temperature-sensing junction 29-1 is at the desired location from needle tip 13-1. Due to the fixed relationship between sensors 21, each successive temperature-sensing junction 29 is therefore properly disposed at its specified position relative to needle tip 13-1.

With the pre-assembled sheath 23 disposed at its proper position within needle 13, UV-cured adhesive is then applied to the open end 13-2 of needle 13 to prevent the resultant assembly from moving therewithin. In this capacity, the resultant assembly effectively forms a thermal well. If required, the outer diameter of needle 13 can then be reduced through a swaging operation.

With the exposed electrical leads of sensors 21 pulled taut, end 13-2 of needle 13 is fittingly inserted into proximal end 33 of hub 17. Thereafter, the interior of hub 17 is preferably filled with UV-cured adhesive to encapsulate and further protect the insulated pair of wires 25 for each sensor 21. As can be appreciated, the adhesive material utilized in the manufacture of needle assembly 11 is preferably selected to ensure proper sterilization and biological compatibility with the intended medical device application.

Referring back to FIGS. 1(a) and (b), the majority of the exposed section of insulation 27 for sensors 21 is fed into a heat shrink tube 37 to further protect wires 25 from any accidental damage as well as to simplify, or manage, the arrangement of the plurality of individual sensor cables.

Finally, the exposed, or free, ends of lead wires 25 for sensors 21-1 thru 21-3 are inserted into the appropriate receptacles of designated electrical connectors 19-1 thru 19-3, respectively. In this manner, each connector 19 is adapted for electrical coupling, either directly or indirectly, to a device capable of converting electrical voltage measurements into corresponding temperature data.

In the present embodiment, connector 19 is represented as comprising a male-type interface, or plug, 39 and a corresponding retention cable clamp 41. However, it is to be understood that connector 19 is not limited to the aforementioned construction and could be replaced with other known types of electrical connectors (e.g. female-type connectors) without departing from the spirit of the present invention.

The invention described in detail above is intended to be merely exemplary and those skilled in the art shall be able to make numerous variations and modifications to it without departing from the spirit of the present invention. All such variations and modifications are intended to be within the scope of the present invention as defined in the appended claims.

What is claimed is:

1. A needle assembly, comprising:
(a) an elongated, hollow needle having a first end and a second end; and
(b) a temperature measurement device at least partially disposed within the needle, the temperature measurement device comprising a plurality of thermal sensors, each thermal sensor comprising a temperature-sensing region;
(c) wherein the temperature-sensing regions for the plurality of thermal sensors are located within the hollow needle at different positions relative to the first end.

2. The needle assembly as claimed in claim 1 further comprising a tubular sheath into which the plurality of thermal sensors is coaxially disposed.

3. The needle assembly as claimed in claim 2 wherein the temperature-sensing regions for the plurality of thermal sensors is located within the tubular sheath.

4. The needle assembly as claimed in claim 3 wherein the tubular sheath is constructed of a dielectric material.

5. The needle assembly as claimed in claim 1 wherein the temperature-sensing regions for the plurality of thermal sensors are equidistantly spaced apart.

6. The needle assembly as claimed in claim 1 wherein the plurality of thermal sensors is arranged in parallel.

7. The needle assembly as claimed in claim 6 wherein the plurality of thermal sensors is secured together with an adhesive.

8. The needle assembly as claimed in claim 1 wherein each thermal sensor generates an electrical voltage in proportion to the temperature sensed within the temperature-sensing region.

9. The needle assembly as claimed in claim 8 wherein each thermal sensor comprises a pair of sensor wires of dissimilar metals.

10. The needle assembly as claimed in claim 9 wherein at least a portion of the pair of sensor wires is dielectrically separated by a layer of insulation.

11. The needle assembly as claimed in claim 10 wherein the pair of sensor wires for each thermal sensor is conductively coupled together to form the temperature-sensing region.

12. The needle assembly as claimed in claim 1 wherein the first end of the needle is in the form of a sharpened tip.

13. The needle assembly as claimed in claim 12 wherein the second end of the needle is in the form of an open port.

14. The needle assembly as claimed in claim 13 wherein the needle further includes a longitudinal, interior channel dimensioned to receive at least a portion of the temperature measurement device.

15. The needle assembly as claimed in claim 1 further comprising a plurality of electrical connectors, each of the plurality of electrical connectors being electrically coupled to one of the plurality of thermal sensors.

16. The needle assembly as claimed in claim 1 further comprising a needle hub mounted on the second end of the needle.
